(19) United States
(12) Patent Application Publication
(10) Pub. No.: US 2020/0196871 A1
(43) Pub. Date: Jun. 25, 2020

(54) DIAGNOSTIC TOOTHBRUSH

(71) Applicants: Wanda Ortiz, Melville, NY (US); Myrna Ortiz, Melville, NY (US)

(72) Inventors: Wanda Ortiz, Melville, NY (US); Myrna Ortiz, Melville, NY (US)

(21) Appl. No.: 16/716,855

(22) Filed: Dec. 17, 2019

Related U.S. Application Data
(60) Provisional application No. 62/784,079, filed on Dec. 21, 2018.

(51) Int. Cl. A61B 5/00; A46B 15/00; A46B 5/00; A46B 13/02; A46B 7/04; A46B 9/04; A61B 1/24; A61B 1/05; A61B 1/00; A61B 8/12

(52) U.S. Cl. CPC A61B 5/0088; A46B 15/004; A46B 5/0095; A46B 13/02; A61B 8/12; A46B 9/04; A61B 1/24; A61B 1/05; A61B 1/00016; A46B 7/04

(57) ABSTRACT

A diagnostic toothbrush comprising, in combination, a toothbrush body, a standard brush head containing toothbrush bristles for brushing teeth, a diagnostic brush head containing a transducer that can detect a dental pathology, and a camera brush head that can produce video images of dental and oral structures. The standard brush head, the diagnostic brush head and the camera brush head can be interchangeably mounted on the toothbrush body. Data from the diagnostic brush head is transferred to the toothbrush body when the diagnostic brush head is mounted on the toothbrush body, data from the camera brush head is transferred to the toothbrush body when the camera brush head is mounted on the toothbrush body, and the data from the diagnostic and camera brush heads is transmitted from the toothbrush body to a computing device.

### CROSS-REFERENCE TO RELATED APPLICATION

The present invention claims priority to U.S. Provisional Application Ser. No. 62/784,079, filed Dec. 21, 2018, the entire disclosure of which is incorporated herein by reference.

### FIELD OF THE INVENTION

The present invention is related to oral health care and specifically, to an apparatus that aids in early, at-home detection of caries, periodontal disease, required root canals, other oral/dental infections and the like.

### BACKGROUND OF THE INVENTION

Good oral hygiene requires periodic examination for, and treatment of, dental caries and other dental pathologies, oral tumors and lesions, parulis, periodontal disease and other oral health issues. Unfortunately, for many people it may be difficult to schedule and/or attend examinations at a dental office due to scheduling conflicts, inconvenience or expense.

### OBJECTS AND SUMMARY OF THE INVENTION

It is therefore an object of the invention to facilitate the examination and diagnosis of dental caries and other dental pathologies, oral tumors and lesions, parulis, periodontal disease and other oral health issues, by adapting a standard electric toothbrush to accommodate brush heads that include sensors for detecting a variety of pathologies, and to transmit data from such brush heads to a dental or medical office located remotely from the user of the toothbrush. The medial or dental professional is thus able to view the output of the brush heads, and diagnose and identify issues without the patient leaving their home. Such brush heads may individually incorporate, for example, a diagnostic transducer, such as an ultrasonic or near-infrared transillumination transmitter/receiver for detecting dental caries, or a camera for detecting oral lesions.

The present invention thus allows the earliest possible diagnosis and identification of an oral/dental health issue and consultation with a medical/dental professional.

In addition, the standard brush head may be equipped with a pressure sensor to monitor and provide feedback to the user if an inappropriate level of pressure is being used during brushing.

In accordance with an aspect of the invention, a diagnostic toothbrush comprises, in combination, a toothbrush body, a standard brush head containing toothbrush bristles for brushing teeth, a diagnostic brush head containing a transducer that can detect a dental pathology, and a camera brush head that can produce video images of dental and oral structures. The standard brush head, the diagnostic brush head and the camera brush head can be interchangeably mounted on the toothbrush body. Data from the diagnostic brush head is transferred to the toothbrush body when the diagnostic brush head is mounted on the toothbrush body, data from the camera brush head is transferred to the toothbrush body when the camera brush head is mounted on the toothbrush body, and the data from the diagnostic and camera brush heads is transmitted from the toothbrush body to a computing device.

The transducer in the diagnostic brush head may be an ultrasonic transmitter/receiver, or a near-infrared translumination device. The computing device may be a computer, a tablet or a smart phone. The toothbrush body may further include a three-dimensional orientation sensor, and data from the orientation sensor may be transmitted from the toothbrush body to the computing device to thus help determine the location of the data from the diagnostic brush head and the camera brush head. The data from the diagnostic brush head and the data from the camera brush head may be transferred from the toothbrush body to the computing device by a USB port, a Bluetooth interface, or a Wi-Fi interface.

### BRIEF DESCRIPTION OF THE DRAWINGS

These and other objects and aspects of the invention will be described with reference tot the following drawing figures, of which:

FIG. 1 is a perspective view of the toothbrush body, having a standard brush head mounted thereon;

FIG. 2 is a diagram of a diagnostic brush head for use with the toothbrush body in accordance with an aspect of the present invention;

FIG. 3 is a diagram of a camera brush head for use with the toothbrush body in accordance with another aspect of the present invention; and

FIG. 4 is an illustration of the toothbrush body of the present invention showing internal components of the body and transmission of data to the computing device.

### DETAILED DESCRIPTION OF THE PREFERRED EMBODIMENTS

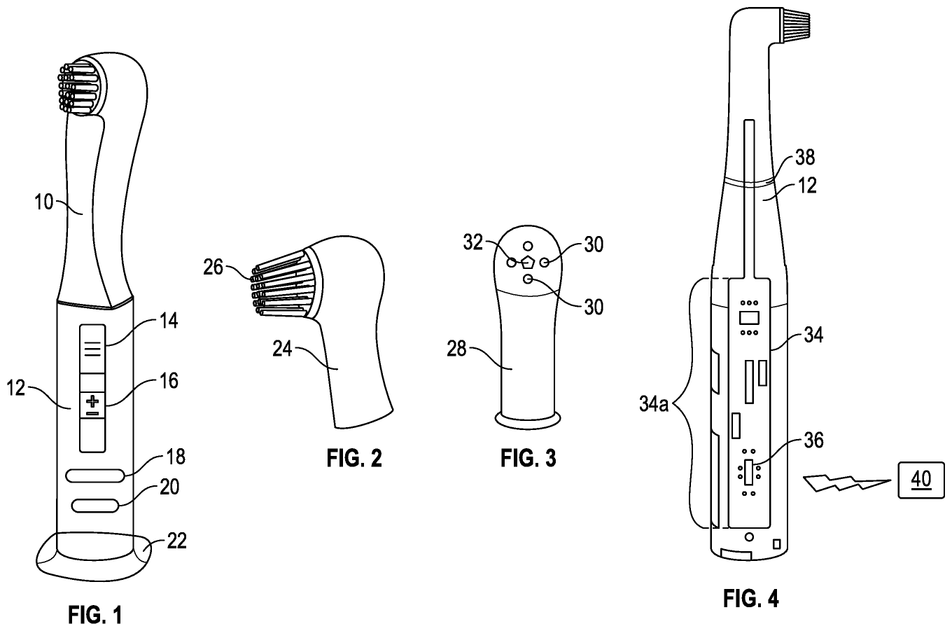

As shown in FIG. 1, a standard brush head 10 is connected to toothbrush body 12. The toothbrush body in accordance with the present invention can accept any one of the standard brush head, a diagnostic brush head, and example of which is shown in FIG. 2, or a camera brush head, an example of which is shown in FIG. 3, as will be described. The brush body 12 is preferably provided with a menu 14 display/input formed of an LED or LCD touch screen display (although a selection input device separate from the display can be used if desired), a feedback display 16 for delivering information to the user in real time about a selected parameter, such as brush pressure or caries detection, a speaker 18 for providing an auditory signal in conjunction with display 16, and on/off switch 20. The body 12 rests in a cradle or base 22 to charge the body when not in use, in a well-known manner.

An example of a diagnostic brush head 24 is shown in FIG. 2, and includes a transducer 26 which may comprise an ultrasonic transmitter/receiver, such as the one disclosed in “A New Diagnostic Tool for Detecting Dental Caries and Tooth Wear: Ultrasonic System,” by Bora Korkut, International Journal of Scientific Research, August 2016 (available at https://www.researchgate.net/publication/307578411) or an optical device such as the near-infrared translumination device described in the “DEXIS CariVu™ Caries Detection device (available at https://www.kavo.com/en-us/imaging-solutions/dexis-carivu-caries-detection), although other diagnostic brush heads may be used in accordance with the present invention. The disclosures of both of the above references are hereby incorporated by reference.

An example of a camera brush head 28 is shown in FIG. 3, and includes one or more light sources 30, which may be LEDs, and a camera lens 32. An example of such camera may be the DEXIS™ DEXcam 4 HD High Definition Intraoral Video Camera, by KaVo Dental (available at https://www.kavo.com/en-us/imaging-solutions/dexis-dexcam-4-hd-intraoral-cameras), although other cameras may be used in accordance with the present invention. The disclosure of the above reference is hereby incorporated by reference.

In the event the user wishes to use the diagnostic brush head, they will replace the standard brush head 10 with the diagnostic brush head 24, and similarly, if the user wishes to use the camera brush head 28, they will replace the standard brush head 10 with the camera brush head.

Operation of the toothbrush, and communications between the diagnostic brush head and/or camera brush head and the dentist or other health care provider, are achieved through use of the components in the toothbrush body, as will be described in connection with FIG. 4. A circuit board 34 is located within the body 12, and includes components 34a providing the toothbrush with the following functionality. For example, in addition to standard functionality, such as charging circuitry, timers, motor control, pressure sensor, and the like, the circuit board is provided with an A/D converter, a video processor for capturing video from the brush head camera 28 and converting it to an appropriate format for transmission to a medical/dental professional, signal amplifiers, sound chip, a 3-D orientation sensor 36 (such as the MotionNode sensor available from Motion Workshop in Seattle, Wash. (https://www.motionnode.com/), the disclosure of which is incorporated by reference), display drivers, and communication apparatus, such as a USB port and/or a Bluetooth and/or Wi-Fi antenna, all of which function in a well-known manner to allow communication between the toothbrush body and a remote device. A microprocessor and associated memory are also provided on circuit board 34 to control the forgoing devices and functionality, and to receive and store data, in a standard manner. Finally, an interface 38 is provided for transmitting data and control signals between the brush heads and the toothbrush body.

In operation, the user will select the appropriate brush head from the menu 14 (or separate input switch) and insert the selected brush head into the body 12. Alternatively, the brush heads can include identifying indicia that allow the body 12 to automatically detect the type of brush head being used. If the standard brush head 10 is selected, a standard brushing cycle is initiated, with feedback from the speaker 20 and/or display 16, if desired, indicating that the user is pressing too hard on their teeth, which can cause tooth erosion or gum recession. If the diagnostic brush head 24 is selected, the user will apply the brush head to their teeth and data from the brush head, which may indicate dental caries and/or other dental pathologies, is generated and delivered to the circuit board 34 via interface 38. Similarly, if the camera brush head is selected, the user will apply the brush head to their teeth and/or other parts of their mouth, and video data from the brush head, which may show dental caries and other dental pathologies, oral tumors and lesions, parulis, periodontal disease and other oral health issues, is generated and delivered to the circuit board 34 via interface 38. In addition to the data generated from the diagnostic and camera brush heads, data from the orientation sensor 36 is also used by the microprocessor to indicate from which area of the mouth the data came. The data from the brush heads and orientation sensor are then transmitted by the USB port or Bluetooth or Wi-Fi antenna to a computing device 40, which may be a standard computer, tablet or smart phone. The computer, tablet or smart phone can be provided with an app in a well-known manner to transmit the data to a dental or medical healthcare provider who can make a preliminary diagnosis of any potential problems by reviewing the received information. If indicated, the healthcare provider can inform the user of any potential issues, at which time an appropriate follow-up can be determined.

Thus, the present invention allows the earliest possible diagnosis and identification of potential health issues, and consultation with a medical/dental professional, without the patient leaving their home.

The foregoing preferred embodiments described herein are set forth as exemplary, and it will be appreciated that various modifications may be made without departing from the scope and spirit of the invention which is defined by the following claims.

What we claim is:

1. A diagnostic toothbrush, comprising, in combination:
a. a toothbrush body;
b. a standard brush head containing toothbrush bristles for brushing teeth;
c. a diagnostic brush head containing a transducer that can detect a dental pathology; and
d. a camera brush head that can produce video images of dental and oral structures;
wherein
i. said standard brush head, said diagnostic brush head and said camera brush head can be interchangeably mounted on said toothbrush body;
ii. data from said diagnostic brush head is transferred to said toothbrush body when said diagnostic brush head is mounted on said toothbrush body;
iii. data from said camera brush head is transferred to said toothbrush body when said camera brush head is mounted on said toothbrush body; and
iv. said data from said diagnostic brush head and camera brush head is transmitted from said toothbrush body to a computing device.

2. The diagnostic toothbrush of claim 1, wherein said transducer in said diagnostic brush head is an ultrasonic transmitter/receiver.

3. The diagnostic toothbrush of claim 1, wherein said transducer in said diagnostic brush head is a near-infrared translumination device.

4. The diagnostic toothbrush of claim 2, wherein said computing device is a computer, a tablet or a smart phone.

5. The diagnostic toothbrush of claim 3, wherein said computing device is a computer, a tablet or a smart phone.

6. The diagnostic toothbrush of claim 1 wherein said toothbrush body includes a three-dimensional orientation sensor, and data from said orientation sensor is transmitted from said toothbrush body to said computing device to thus help determine the location of said data from said diagnostic brush head and said camera brush head.

7. The diagnostic toothbrush of claim 1 wherein said data from said diagnostic brush head and said data from said camera brush head are transferred from said toothbrush body to said computing device by a USB port.

8. The diagnostic toothbrush of claim 1 wherein said data from said diagnostic brush head and said data from said camera brush head are transferred from said toothbrush body to said computing device by a Bluetooth interface.

9. The diagnostic toothbrush of claim 1 wherein said data from said diagnostic brush head and said data from said camera brush head are transferred from said toothbrush body to said computing device by a Wi-Fi interface.
